(19) United States
(12) Patent Application Publication
(10) Pub. No.: US 2004/0121447 A1
(43) Pub. Date: Jun. 24, 2004

(54) Method and apparatus for concentrating an aqueous suspension of microalgae

(76) Inventor: Real Fournier, Rimouski (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/703,150

(22) Filed: Nov. 6, 2003

(30) Foreign Application Priority Data
Nov. 7, 2002 (CA) 2,411,383,

(51) Int. Cl. C12N 1/12

(52) U.S. Cl. 435/257.1

(57) ABSTRACT

The invention relates to a method and apparatus for concentrating an aqueous suspension of microalgae. The suspension of microalgae is passed through a tangential filtering device for partially removing water from the suspension without rupturing the microalgae, thereby obtaining a concentrated suspension of microalgae and filtered water. Such a method can be use in systems for production of microalgae. An apparatus for carrying out the method according to the invention is also disclosed.

### FIELD OF THE INVENTION

The present invention relates to improvements in the field of the production of microalgae. More particularly, the invention relates to an improved method and apparatus for concentrating an aqueous suspension of microalgae.

### BACKGROUND OF THE INVENTION

Microalgae are at the basis of the marine alimentary chain. For many marine organisms, microalgae represent the sole source of food. The culture of zooplankton and mollusk requires a massive production of microalgae. It is generally admitted that the production costs of microalgae represent about one third of the operation costs of a commercial hatchery. Much research has been done in order to develop an alternative diet which may totally or partially replace a natural diet consisting of feeding the marine microorganism with natural food. These alternative diets have been proposed in order to reduce and even to eliminate the high production costs of the microalgae. Microalgae paste was one of the suggested alternative diets to replace diets consisting of living microalgae. These pastes are prepared by centrifugation or flocculation processes for obtaining concentrated suspension of microalgae. The major drawback of the methods of preparing concentrated suspension of microalgae is that the obtained microalgae have a low nutritive value. This considerable loss is explained by the fact that even if such techniques are efficient for concentrating and preserving the algal biomass, they do not allow the preservation of living biological material. In fact, when using such methods, a rapid biochemical degradation of the microalgae occurs. In particular, the lipidic content of the microalgae is substantially reduced. Thus, the microalgae paste and other substitutes such as microencapsulated lipids and microalgae powders cannot completely replace natural diets consisting of living microalgae.

U.S. Pat. No. 5,910,254 describes a method for dewatering an aqueous suspension of microalgae by introducing the suspension into a bubble column for generating a froth of bubbles and adsorbed algal cells that can be separated from the aqueous suspension. This method permits to isolate valuable organic compounds from microalgae such as beta carotene, carotenoids, glycerol and proteins, but does not maintain the integrity of the microalgae since the latter are ruptured during the method.

U.S. Pat. No. 6,524,486 describes a method and apparatus for separating microalgae from water without rupturing cells. Such a method comprises three different steps (flocculation, flotation and dehydration) and requires the use of flocculating agents.

When using flocculating agents or preservative agents, chemicals are added to the concentrated suspension of microalgae and the effects of these products on the stability of the suspension are often unknown.

Many pharmaceutical and neutraceutical products are supplied from the environment, such as animals, plants, bacteria and fungus. Also, a plurality of new bioactive molecules have been extracted and isolated from marine organisms. It has been estimated that about 30,000 different species of microalgae are present in the ocean. One of the biggest challenges is thus to facilitate the supply of these microorganisms. Even if the industrial production of microalgae has been required for the aquaculture for decades, recuperation of the vegetal biomass for the eventual extraction of a new bioactive molecule is quite recent. Since the methods used so far for extracting and isolating microalgae from their culture mediums (centrifugation and flocculation) and their preservation (freezing and preservatives) are known to reduce the quality of the obtained microalgae, it is evident that the development of new methods is needed.

### SUMMARY OF THE INVENTION

It is therefore an object of the present invention to overcome the above drawbacks and to provide a method and apparatus for concentrating a suspension of microalgae without rupturing the microalgae.

According to a first aspect of the invention, there is provided a method of concentrating an aqueous suspension of microalgae, comprising the step of passing the suspension of microalgae through a tangential filtering device for partially removing water from the suspension without rupturing the microalgae, thereby obtaining a concentrated suspension of microalgae and filtered water.

According to a second aspect of the invention, there is provided a method of producing a concentrated suspension of microalgae, comprising the steps of:

a) providing a reservoir containing an aqueous suspension of microalgae, and a tangential filtering device in fluid flow communication with the reservoir;

b) passing the suspension from the reservoir through the tangential filtering device to partially remove water from the suspension without rupturing the microalgae, thereby obtaining the concentrated suspension of microalgae and filtered water; and

c) recovering the concentrated suspension of microalgae.

According to a third aspect of the invention, there is provided an apparatus for concentrating an aqueous suspension of microalgae, comprising:

a reservoir dimensioned to contain the suspension of microalgae to be concentrated;

a tangential filtering device in fluid flow communication with the reservoir, for partially removing water from the suspension without rupturing the microalgae; and

a pump for passing the suspension from the reservoir through the tangential filtering device, thereby obtaining a concentrated suspension of microalgae and filtered water.

Applicant has found quite surprisingly that by using a tangential filtering device for partially removing water from the aqueous suspension of microalgae, it is possible to concentrate the suspension of microalgae without rupturing the microalgae.

The expression “microalgae in the concentrated suspension obtained have a reproductive potential which is maintained for a period of at least 25 days” as used herein means that over a period of 25 days, the reproductive potential of the microalgae permits a constant growth of a culture of these microalgae.

### DETAILED DESCRIPTION OF THE INVENTION

In the method according to the first aspect of the invention, the suspension prior to being concentrated can have a concentration ranging from 1 to 500×10⁶ cells/mL and preferably from 1×10⁶ to 50×10⁶ cells/mL. In the method according to the second aspect of the invention, the suspension prior to being concentrated can have a concentration ranging from 1 to 100×10⁶ cells/mL and preferably from 1×10⁶ to 30×10⁶ cells/mL. The suspension prior to being concentrated according to the methods of the invention can originate from a fresh culture of microalgae.

The concentrated suspension obtained according to the method as defined in the first aspect of the invention can have a concentration ranging from 2 to 30×10¹⁰ cells/mL and preferably from 2×10⁶ to 10×10¹⁰ cells/mL. The concentrated suspension obtained according to the method as defined in the second aspect of the invention can have a concentration ranging from 1×10⁶ to 30×10¹⁰ cells/mL and preferably from 2×10⁶ to 10×10¹⁰ cells/mL.

The concentrated suspension obtained according to the methods of the invention can be from 2 to 1000 and preferably from 100 to 800 times more concentrated than the suspension prior to concentration.

The filtered water obtained in step (b) according to the methods of the invention can be used for the culture of microalgae.

The method as defined in the second aspect of the invention can further include prior to step (c):

b′) recycling the concentrated suspension obtained in step (b) to the reservoir and then repeating step (b).

Preferably, step (b′) is repeated until the suspension obtained reaches a desired concentration. The desired concentration can range from 1×10⁶ to 30×10¹⁰ cells/mL and preferably from 2×10⁶ to 10×10¹⁰ cells/mL or can be from 4 to 1000 and preferably from 100 to 800 times more concentrated than the suspension prior to concentration. During step (b) or (b′), a fresh suspension of microalgae can be added into the reservoir. Step (c) can be carried out by recovering the concentrated suspension of microalgae from the reservoir. Preferably, step (c) is carried out by recovering the concentrated suspension of microalgae from the reservoir and from the tangential filtering device.

The method according to the first aspect of the invention can further comprise the step of recovering the concentrated suspension of microalgae. The methods of the invention are preferably continuous methods.

In the methods of the invention, the step of passing the suspension through the tangential filtering device can be an ultrafiltration.

In the methods of the invention and in the apparatus according to the third aspect of the invention, the tangential filtering device can comprise a cartridge containing a plurality of spaced-apart parallel tubular members, wherein the tubular members have porous walls with pores of a predetermined molecular weight cut-off.

In the methods of the invention and in the apparatus according to the third aspect of the invention, the tangential filtering device can comprise a plurality of tangential filtration cartridges arranged in fluid flow communication with one another or in parallel relationship to one another. Preferably, the tangential filtration cartridges each contain a plurality of spaced-apart parallel tubular members, wherein the tubular members have porous walls with pores of a predetermined molecular weight cut-off.

The molecular weight cut-off of the pores of the tubular member, in the methods of the invention and in the apparatus according to the third aspect of the invention, can range from 1000 to 100000 Daltons and preferably from 5000 to 20000 Daltons. The tubular members are preferably hollow fibers. The tubular members can define a total filtration surface ranging from 0.03 to 300 m², preferably from 5 to 130 m² and even more preferably from 10 to 25 m².

In the methods of the invention, the suspension passing through the tangential filtering device can have a flow rate ranging from 1 to 5000, preferably from 100 to 1000 and more preferably from 250 to 500 L/hour. The pressure of the suspension passing through the tangential filtering device can range from 1 to 150 psi and preferably from 5 to 25 psi. The tangential filtering device can be disposed vertically and the suspension is passed therethrough upwardly or they can be disposed horizontally.

The microalgae in the methods and the apparatus of the invention can be marine or freshwater microalgae. The microalgae can be selected from the group consisting of non-motile unicellular algae, flagellates, diatoms and blue-green algae. The microalgae can belong to the family of Chlorophyceae, Prasinophyceae, Bacillariophyceae, Cryptophyceae, Chrysophycea, Haptophyceae or Cyanophyceae. The microalgae can belong to a species selected from the group consisting of Isochrysis galbana, Monochrysis lutheri, Chaetoceros muelleri and Nannochloropsis sp. The microalgae can have a size ranging from 1 to 100 μm and preferably from 3 to 20 μm.

In the methods of the invention, the microalgae in the concentrated suspension obtained can have a lipidic content which is stable for at least 30 days, preferably for at least 15 days and more preferably for at least 12 days. The microalgae in the concentrated suspension can have a phospholipid content or cholesterol content which is stable for at least 30 days, preferably for at least 15 days and more preferably for at least 12 days. The microalgae in the concentrated suspension obtained can have a reproductive potential which is maintained for a period of at least 25 days. The microalgae in the concentrated suspension obtained can have a reproductive potential similar to fresh microalgae for a period of at least 30 days, preferably for at least 15 days and more preferably for at least 12 days.

In the methods of the invention, the suspension prior to concentration and the concentrated suspension obtained can have similar lipidic contents. The suspension prior to concentration and the concentrated suspension obtained preferably have similar phospholipid contents, similar cholesterol contents or similar nutritive values. The nutritive value of the microalgae in the concentrated suspension obtained can be maintained for at least 30 days and preferably for at least 15 days. Preferably, the microalgae in the concentrated suspension obtained are alive.

In the apparatus according to the third aspect of the invention, the reservoir can have a capacity ranging from 1 to 5000 L and preferably from 100 to 500 L. The Pump can be adapted to impart to the suspension a flow rate ranging from 1 to 5000 L/hour and preferably from 100 to 500 L/hour, or a pressure ranging from 1 to 150 psi and preferably from 5 to 25 psi.

The cartridge in the apparatus according to the third aspect of the invention can have a feed inlet for receiving the suspension of microalgae to be concentrated, a first outlet for discharging the filtered water and a second outlet for discharging the concentrated suspension of microalgae, wherein the tubular members define therebetween a space in fluid flow communication with the first outlet, each the tubular member having an inlet in fluid flow communication with the feed inlet and an outlet in fluid flow communication with the second outlet. The second outlet can be connected to the reservoir by a first conduit for recycling the concentrated suspension discharged from the cartridge. The feed inlet can be connected to the reservoir by a second conduit. Preferably, the first and second conduits are connected together by a third conduit.

The first outlet in the apparatus according to the third aspect of the invention is preferably connected to a drain by a fourth conduit. The first conduit and the fourth conduits are preferably connected together. The second conduit can be provided with a drain for emptying the reservoir or for emptying the cartridge. The first conduit can provided with a flow control device for controlling the flow rate of the concentrated suspension discharged from the cartridge. The second conduit can be provided with a flow control device for controlling the flow rate of the suspension passing through the tangential filtering device. The pump is preferably disposed between the reservoir and the cartridge, in the second conduit.

In the apparatus according to the third aspect of the invention, when the tangential filtration cartridges contain a plurality of spaced-apart parallel tubular members, each cartridge preferably has a feed inlet for receiving the suspension of microalgae to be concentrated, first outlet for discharging the filtered water and second outlet for discharging the concentrated suspension of microalgae, wherein the tubular members define therebetween a space in fluid flow communication with the first outlet, each the tubular member having an inlet in fluid flow communication with the feed inlet and an outlet in fluid flow communication with the second outlet.

The concentrated suspension of microalgae obtained by the methods of the invention can be useful for extracting and/or isolating bioactive molecules. The concentrated suspension of microalgae obtained by the methods of the invention can also used for feeding marine organisms. The marine organisms can be zooplanktons and preferably copepods. The marine organisms can also be mollusks and preferably filter feeding mollusks. The methods and the apparatus of the invention can be useful in a system for feeding marine organisms, in a system for producing microalgae as food for marine organisms, in a system for producing microalgae as a health food, in a system for producing microalgae as a biofuel, in a system for producing microalgae for extracting and/or isolating bioactive molecules or in a system for producing microalgae for pharmaceutical use.

### BRIEF DESCRIPTION OF THE DRAWINGS

Further features and advantages of the invention will become more readily apparent from the following description of preferred embodiments as illustrated by way of examples in the accompanying drawings, in which:

FIG. 1 is a schematic representation of an apparatus for concentrating a suspension of microalgae, according to a preferred embodiment of the invention;

FIG. 2 is a schematic representation of an apparatus for concentrating a suspension of microalgae, according to another preferred embodiment of the invention;

FIG. 3 is a sectional elevation view of the tangential filtration cartridge shown in FIG. 1;

FIG. 4 is a sectional view taken along line 4-4 of FIG. 3;

FIG. 5 is a graph showing the evolution of the reproductive potential of microalgae from a concentrated suspension of microalgae obtained according to a method of the invention; and

FIG. 6 is a schematic representation of an apparatus for concentrating a suspension of microalgae, according to still another preferred embodiment of the invention.

### DESCRIPTION OF PREFERRED EMBODIMENTS

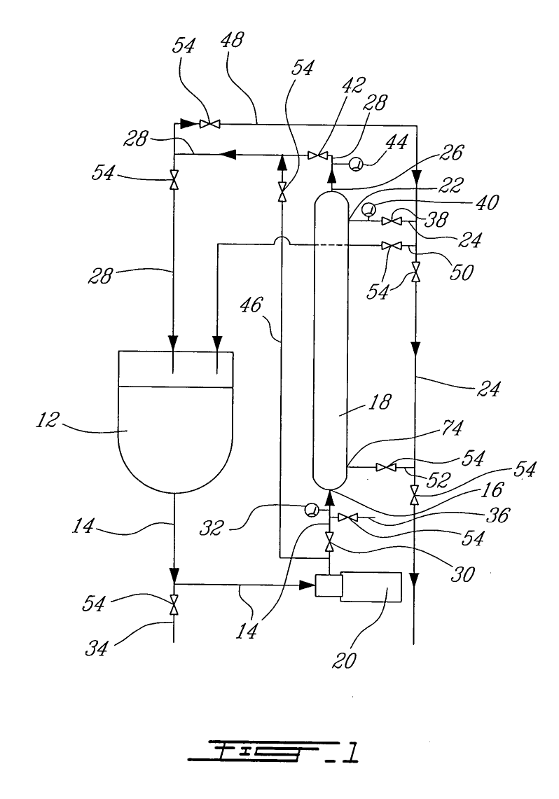

Referring first to FIG. 1, there is illustrated an apparatus for concentrating an aqueous suspension of microalgae, wherein a suspension of microalgae contained in a reservoir 12 is supplied or conveyed via conduit 14 to the inlet 16 of a tangential filtration cartridge 18 by means of pump 20. The suspension of microalgae is passed through the tangential filtration cartridge 18 where it is concentrated, thereby obtaining filtered water which is discharged via outlet 22 and supplied via conduit 24 to a drain (not shown), and a concentrated suspension of microalgae which is discharged via outlet 26 and supplied via conduit 28 to the reservoir 12 for optionally being further concentrated. The conduit 14 is provided with a valve 30 for controlling the flow rate of the suspension passing through the cartridge 18, and with a manometer 32 which indicates the pressure generated by the flow rate of the suspension to be concentrated. A conduit 34 is connected to conduit 14 for emptying the reservoir 12. A conduit 36 is also connected to conduit 14 for emptying the cartridge 18. The conduit 24 is provided with a valve 38 for controlling the flow rate of the filtered water discharged from the cartridge 18, and with a manometer 40 which indicates the pressure generated by the flow rate of the filtered water. The conduit 28 is provided with a valve 42 for controlling the flow rate of the concentrated suspension discharged from the cartridge 18, and with a manometer 44 which indicates the pressure generated by the flow rate of the concentrated suspension.

Conduits 14 and 28 are connected together by conduit 46, and conduits 24 and 28 are connected together by a conduit 48. Conduits 46 and 48 are used for bypassing the inlet 16 of the cartridge 18 when recovering the concentrated suspension obtained. For recovering the concentrated suspension obtained, filtered water is introduced into the reservoir 12 and supplied to the outlet 22 via conduits 14, 46, 28, 48 and 24. The filtered water is then passed through the cartridge 18 downwardly. The recovered concentrated suspension is then discharged via conduit 36.

The tangential filtration cartridge 18 is provided with an outlet 74 which is connected to the conduit 24 by a conduit 52. The outlet 74 and conduit 52 are used only for draining the cartridge 18, when the cartridge 18 is cleaned. Conduit 24 is connected to the reservoir 12 by a conduit 50. The conduit 50 is used when filtered water is supplied via conduits 48 and 24 for cleaning the apparatus. Conduits 24, 28, 34, 36, 46, 48, 50 and 52 are each provided with a flow rate controlling valve 54.

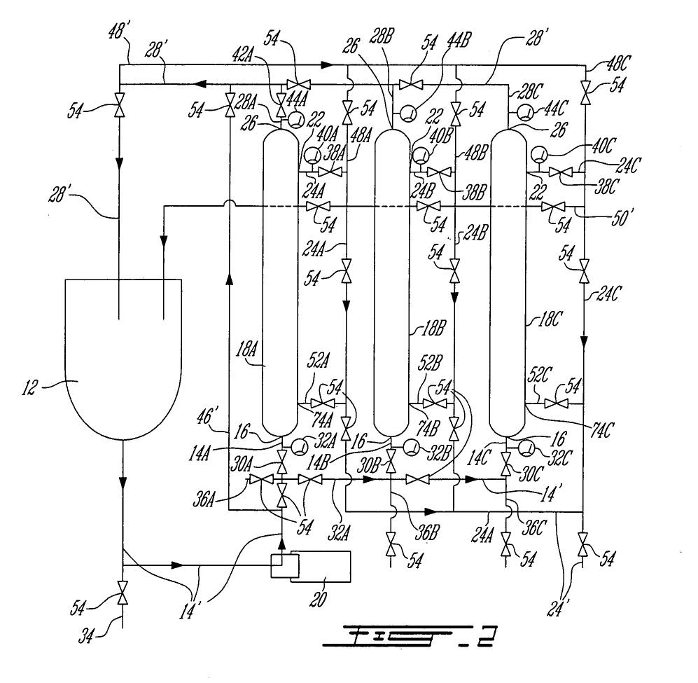

Referring to FIG. 2, three tangential filtration cartridges 18A,18B,18C are identical to the tangential filtration cartridge 18 shown in FIG. 1 and are arranged in parallel relationship to one another. An aqueous suspension of microalgae contained in the reservoir 12 is supplied via a common conduit 14′ and then via conduits 14A, 14B and 14C to the inlets 16 of tangential filtration cartridges 18A, 18B and 18C by means of pump 20, for being concentrated. The suspension of microalgae is then passed through the tangential filtration cartridges 18A, 18B and 18C where it is concentrated, thereby obtaining filtered water which is discharged via outlets 22 and supplied via conduits 24A, 24B and 24C to a common conduit 24′ and then to a drain (not shown). The concentrated suspension of microalgae obtained is discharged via outlets 26 of cartridges 18A, 18B and 18C, and supplied via conduits 28A, 28B and 28C to a common conduit 28′ and then to the reservoir 12 for optionally being further concentrated. The conduits 14A, 14B and 14C are provided with valves 30A, 30B and 30C for controlling the flow rate of the suspension passing through the cartridges 18A, 18B and 18C, and with manometers 32A, 32B and 32C which indicate the pressure generated by the flow rate of the suspension to be concentrated. Conduit 34 is connected to conduit 14′ for emptying the reservoir 12. Conduits 36A, 36B and 36C are connected to conduits 14A, 14B and 14C for emptying the cartridges 18A, 18B and 18C. The conduits 24A, 24B and 24C are provided with valves 38A, 38B and 38C for controlling the flow rate of the filtered water discharged from cartridges 18A, 18B and 18C, and with manometers 40A, 40B and 40C which indicate the pressure generated by the flow rate of the filtered water. The conduit 28A is provided with a valve 42A for controlling the flow rate of the concentrated suspension discharged from the cartridge 18A. The conduits 28A, 28B and 28C are provided with manometers 44A, 44B and 44C which indicate the pressure generated by the flow rate of the concentrated suspension discharged from cartridges 18A, 18B and 18C.

Conduits 14′ and 28′ are connected together by conduit 46′, and conduits 24A, 24B and 24C are connected to conduit 28′ by a combination of conduit 48′ with conduits 48A, 48B and 48C. Conduits 46′ and 48′ are used for bypassing the inlets 16 of the cartridges 18A, 18B and 18C when recovering the concentrated suspension obtained. For recovering the concentrated suspension obtained, filtered water is introduced into reservoir 12 and supplied to the outlets 22 of cartridges 18A, 18B and 18C via conduits 14′, 46′, 28′, 48′, 48A, 48B, 48C, 24A, 24B and 24C. The filtered water is then passed through the cartridges 18A, 18B and 18C downwardly. The recovered concentrated suspension is then discharged via conduits 14A, 14B, 14C, 36A, 36B and 36C.

The cartridges 18A, 18B and 18C have respective outlets 74A, 74B and 74C which are connected to conduits 24A, 24B and 24C by conduits 52A, 52B and 52C, respectively. The outlets 74A,74B,74C, and conduits 52A,52B,52C are used only as draining means when cleaning the cartridges 18A, 18B and 18C. The conduits 24A, 24B and 24C are connected to the reservoir 12 by a conduit 50′. The conduit 50′ is used when filtered water is supplied via conduits 48A, 48B, 48C, 24A, 24B and 24C for cleaning the apparatus. Conduits 14′, 14A, 14B, 14C, 24A, 24B, 24C, 28′, 34, 36A, 36B, 36C, 46′, 48A, 48B, 48C, 50′, 52A, 52B and 52C are each provided with a control flow rate valve 54.

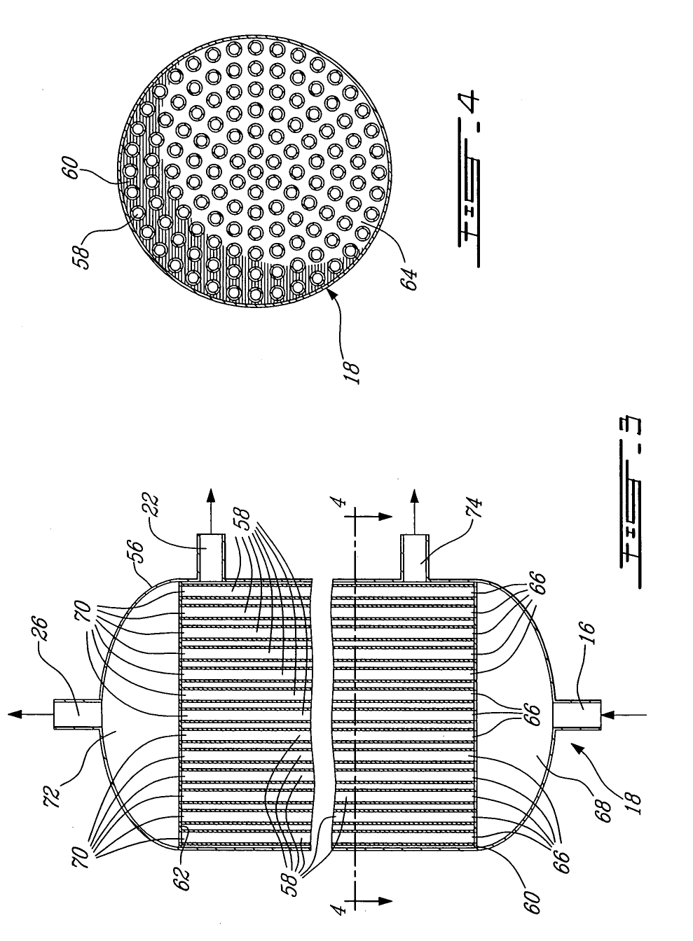

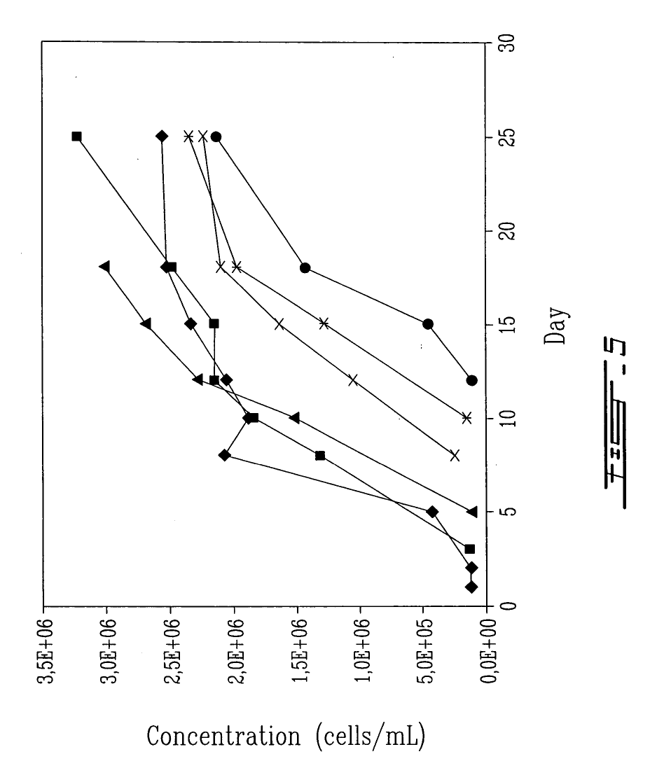

As shown in FIGS. 3 and 4, the tangential filtration cartridge 18 comprises a housing 56 provided with inlet 16 for receiving the aqueous suspension of microalgae to be concentrated, outlet 22 for discharging filtered water, outlet 26 for discharging the concentrated suspension of microalgae obtained and outlet 74 for draining the cartridge 18 when the latter is cleaned. The cartridge 18 further comprises a plurality of hollow fibers 58 arranged in spaced-apart parallel relationship inside the housing 56. The hollow fibers 58 are formed of a porous material and are supported by lower and upper apertured plates 60 and 62. The fibers 58 define therebetween a space 64 (shown in FIG. 4) in fluid flow communication with outlets 22 and 74. Each fibre 58 has an inlet 66 in fluid flow communication with an inlet chamber 68 which in turn is in fluid flow communication with the inlet 16 of the housing 56, and an outlet 70 in fluid flow communication with an outlet chamber 72 which in turn is in fluid flow communication with the outlet 26 of the housing. The inlets 66 and outlets 70 of the hollow fibers 58 register with the apertures formed in plates 60 and 62.

The aqueous suspension of microalgae supplied to the tangential filtration cartridge 18 flows through the inlet 16 and into the chamber 68, and enters each hollow fibre 58 through the inlet 66. A portion of the water passes through the pores defined in the walls of the fibers 58 and is thus filtered, the filtered water being discharged into the space 64. The filtered water is discharged from the cartridge 18 through the outlet 22. The concentrated suspension of microalgae exits the hollow fibers 58 through the outlets 70, flows through the chamber 72 and is discharged from the cartridge 18 through the outlet 26.

The apparatus schematized in FIG. 6 is similar to the apparatus schematized FIG. 1. In fact, the apparatus of FIG. 6 is a simplified version of the apparatus of FIG. 1 wherein conduits 46, 48 and 50 have been removed and wherein valve 30 of conduit 14 and valve 54 of conduit 24 have been replaced with threeway valves 31 and 55, respectively. Moreover, a conduit 37 connected to conduits 14 and 25 has been added.

The following examples given in a non-limitative manner are focused on the methods of the invention using the apparatus schematized in FIG. 1 or FIG. 6.

### EXAMPLE 1

The concentration of various types of microalgae has been carried out using the following general procedures using the apparatus schematized in FIG. 1. At the beginning of the procedure, all the valves were closed. The reservoir 12 has been filled with an aqueous suspension of microalgae to be concentrated. Valves 38 and 42 as well as valves 54 of conduits 24 and 28 have been opened and the pump 20 has been turned on. Then, valve 30 has been opened slowly until a pressure of 5 psi has been obtained on the manometer 32. The cartridge 18 has been filled completely until filtered water has been discharged into the drain. Valve 30 has been further opened until a pressure of 20 psi has been obtained according to the manometer 32. Valve 42 has been slowly turned off in order to generate a pressure of 5-10 psi according to manometer 44. The suspension of microalgae is passed through cartridge 18, discharged via conduit 28 and recycled into the reservoir 12 and eventually passed again through cartridge 18 for further concentration. The suspension to concentrate is circulated into the apparatus until the desired concentration is obtained. When the desired concentration has been obtained, the valve 30 has been slowly and completely turned off. Then, the pump 20 and all the opened valves have also been turned off.

Then, the concentrated suspension of microalgae has been recovered in a container (not shown) by opening valve 54 of conduit 46, and then opening valve 54 of conduit 34 in order to empty reservoir 12. Valves 54 of conduits 34 and 46 have been closed. The reservoir 12 has been filled with about 20 liters of the obtained filtered water or with filtered sea water. A further container (not shown) has been disposed under the conduit 36, and valve 54 of conduit 36 has been opened. Then, valves 54 of conduits 46 and 48 have been opened. The pump has been turned on and valve 38 has been opened in order to generate a pressure lower than 10 psi on manometer 40. The filtered water has been passed downwardly (or counter-current) through cartridge 18 to remove all the concentrated suspension from the hollow fibers of the cartridge 18. The concentrated suspension has been discharged from the cartridge 18 via the conduit 36. When all the concentrated suspension has been removed from the cartridge, valve 38 and then valve 54 of conduit 36 have been closed. Finally, the pump 20 has been turned off.

Finally, the apparatus schematized in FIG. 1 has been cleaned by first opening valve 54 of conduit 34 and rinsing reservoir 12 with fresh water. Then, valve 54 of conduit 34 has been closed and the reservoir 12 has been filed with 20 litres of fresh water. The pump 20 has been turned on and valves 54 of conduits 28 and 46 have been opened. Water has been circulated few seconds and valve 54 of conduit 28 has been closed. Valves 54 of conduits 48 and 50 have been opened and water has been circulated through conduits 48 and 50 for few seconds. Valves 54 of conduits 46, 48 and 50 have then been closed. A drain (not shown) and conduit 36 have been connected together, and valve 42 and valve 54 of conduit 36 have been opened. The valve 54 of conduit 46 has been opened until a pressure of 5 psi was reached on manometer 44. Water has been passed through cartridge 18 for about one minute and valve 42 has been closed. Valve 54 of conduit 24 has been opened and then valve 54 of conduit 48 has been slowly opened until a pressure of 5 psi has been reached on manometer 40. Water has been passing through the cartridge 18 and discharged into the drain until a limpid water has been obtained. Valve 54 of conduit 46 has been closed and the pump 20 has been turned off. Then, all the valves of the apparatus have been opened, the apparatus has been drained and all the valves have been closed. The reservoir has been filled with 20 litres of a cleaning and sterilizing solution such as a solution of 200 ppm of sodium hypochlorite. Valves 38 and 42 as well as valves 54 of conduits 24 and 28 have been opened and the pump 20 has been turned on. Then, valve 30 has been opened slowly until a pressure of 20 psi has been obtained on the manometer 32. The cleaning and sterilizing solution has been passed through the cartridge 18 and then, valve 30 has been closed. The pump 20 has been turned off, all the valves have been opened and the apparatus has been drained and all the valves have then been closed.

### EXAMPLE 2

The concentration of various types of microalgae has also been carried out using the following general procedures using the apparatus schematized in FIG. 6. At the beginning of the procedure, all the valves were closed. The reservoir 12 has been filled with an aqueous suspension of microalgae to be concentrated. Valve 42 as well as valve 54 of conduit 25 have been opened. Valve 55 is opened in such a manner of permitting passage from conduit 24 to conduit 25 and the pump 20 has been turned on. Then, valve 31 has been opened slowly until a pressure of 5 psi has been obtained on the manometer 32. The cartridge 18 has been filled completely until filtered water has been discharged into the conduit 25. Valve 31 has been further opened until a pressure of 20 psi has been obtained according to the manometer 32. Valve 42 has been slowly turned off in order to generate a pressure of 5-10 psi according to manometer 44. The suspension of microalgae is passed through cartridge 18, discharged via conduit 28 and recycled into the reservoir 12 and eventually passed again through cartridge 18 for further concentration. The suspension to concentrate is circulated into the apparatus until the desired concentration is obtained. When the desired concentration has been obtained, the valve 31 has been slowly and completely turned off. Then, the pump 20 and all the opened valves have also been turned off.

Then, the concentrated suspension of microalgae contained in the reservoir 12, conduits 14 and 28, cartridge 18 and pump 20 is recovered in an appropriate container (not shown) through conduit 34 by opening valve 54 of the latter conduit, and then opening valve 31 in such a manner to permit passage from the pump 20 to the cartridge 18. When a maximum amount of the concentrated suspension has been recovered, all valves have been closed. The reservoir 12 has been filled with about 20 liters of the obtained filtered water or with filtered sea water. A further container (not shown) or same has been disposed under the conduit 36, and valve 54 of conduit 36 has been opened. Then, valve 31 has been opened in such a manner to permit passage from the pump 20 to the conduit 37. The pump has been turned on and valve 55 has been opened in such a manner to permit the passage the conduit 37 to the conduit 24, and to generate a pressure lower than 10 psi on manometer 40. The filtered water has been passed downwardly (or counter-current) through cartridge 18 to remove all the concentrated suspension from the porous wall of the hollow fibers of the cartridge 18. The concentrated suspension has been discharged from the hollow fibres of the cartridge 18 via the conduit 36. When all the concentrated suspension has been removed from the cartridge, valve 31 has been closed and the pump 20 has been turned off. Then, all the other valves have been closed.

The apparatus schematized in FIG. 6 has been cleaned and sterilized by first opening valve 54 of conduit 34 and rinsing reservoir 12 with fresh water. Then, valve 54 of conduit 34 has been closed and the reservoir 12 has been filed with at least 20 litres of fresh water. The pump 20 has been turned on and valve 54 of conduit 36 has been opened. The valve 31 is opened in such a manner to permit passage from the pump 20 to the cartridge 18 and by verifying the manometer 32 in order to maintain the pressure below 10 psi. The valve 31 is then close after few seconds. Valve 54 of conduit 25 is opened and valve 55 is opened in such a manner to permit passage from conduit 24 to conduit 25. Valve 31 has then been opened in such a manner to permit passage from the pump 20 to the cartridge 18, until a pressure of 10 psi is obtained on manometer 32. Water has been passing through the cartridge 18 and discharged through conduit 25 until a limpid water has been obtained. Fresh water is further added into the reservoir 12 if needed. Finally, the reservoir is emptied by opening valve 42 and opening valve 31 in such a manner to permit passage from the pump 20 to the conduit 37. Then, valve 31 is closed and the pump 20 is turned off. The valves are all opened and the apparatus is completely drained. The valves 31 and 55 are opened in all possible manners in order to permit draining of the cartridge 18 as well as conduits 24, 36 and 52. Then, all the valves are closed.

The reservoir 12 has been filled with 20 litres of a cleaning and sterilizing solution such as a 200 ppm solution of sodium hypochlorite. Valve 42 is opened and valve 55 is opened in such a manner to permit passage from conduit 37 to conduit 24. The pump 20 has been turned on. The valve 31 is opened in such a manner to permit passage from the pump 20 to conduit 37 until a pressure of 10 psi is obtained on manometer 40. The cleaning and sterilizing solution has been passed through the cartridge 18 for about 10 minutes and then, conduits 25 and 36 are connected to a drain prior to open their valves 54. When the whole has been circulated, the pump 20 has been turned off. All the valves have been opened in all possible manners in order to permit a complete draining of the cartridge 18 and the conduits 36, 37 and 52. Finally, all the valves have been closed.

With respect to the apparatuses schematized in FIGS. 1 and 6, it should be noted that when preparing two (or more) separate batches of concentrated suspension of microalgae within few hours (using the same of microalgae), cleaning of the apparatuses between each batch is not absolutely necessary. The recovering of the concentrated suspension obtained in a batch can be carried out simply by emptying the reservoir 12.

### EXAMPLE 3

Using the above-mentioned general procedure for the apparatus schematized in FIG. 1, aqueous suspensions of microalgae have been concentrated. In particular, suspensions of two different species of microalgae, Isochrysis galbana and Chaetoceros muelleri, have been concentrated. Suspensions of these microalgae varying from 300 to 1000 L have been concentrated from 100 to 500 times. In fact, suspensions having an initial concentration of 15×10⁶ cells/mL have been concentrated until a concentration of about 5×10⁹ to 8×10⁹ cells/mL was obtained. The flow rate of the suspension to concentrate passing through the cartridge was about 300 L/hour. The hollow fibers of the cartridge had a total filtration surface of about 5 to about 13 m².

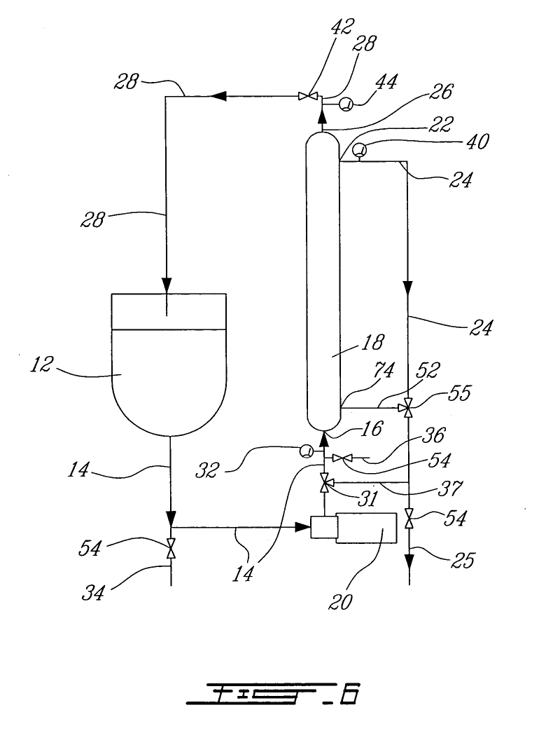

In order to evaluate the quality of the concentrated suspensions of microalgae obtained, two tests have been performed on these suspensions. Firstly, about 500 L of a suspension of a culture of Chaetoceros muelleri having an initial concentration of 12×10⁶ cells/mL has been concentrated to a volume of 4 L. Then, the concentrated suspension has been stocked into darkness at 4° C. Microalgae have been kept in suspension by bubbling the suspension. The concentrated suspension has been kept in such conditions for a period of twelve days. Samples of the suspension have been taken every two days to evaluate the reproductive potential of the microalgae (see FIG. 5). The samples have been prepared by adding two or three drops of the suspensions into test tubes containing a culture medium. The concentration of these cultures has been evaluated with a particle counter until the 25^{th} day after the beginning of the test. As illustrated on FIG. 5, the microalgae of the concentrated suspension obtained maintained their reproductive potential during all the testing period.

Secondly, the cholesterols, photosynthetic pigments and phospholipids contents (or lipidic content) of the concentrated suspension of culture of Chaetoceros muelleri have been evaluated. As demonstrated in Table 1, these contents have not been affected during the 12 days storage of the suspension. It should be noted that some of irregular variations observed in these contents during the period of 12 days seem to occur randomly and are probably related to the extraction and analysis procedures used. An interesting fact is that the phospholipid and the cholesterol contents did not vary substantially during this period. Phospholipids and cholesterols are known to have an important role in the structure of the cellular membrane of the microalgae.

TABLE 1
Evolution of the composition of microalgae
during a 12 days storage

| | | Photosynthetic | | |
|---|---|---|---|---|
| | Cholesterols | pigments | Phospholipids | Total |
| Day | (μg/mL) | (μg /mL) | (μg /mL) | (μg /mL) |
| 0 | 0,265 | 13,252 | 26,063 | 39,580 |
| 2 | 2,434 | 14,530 | 28,364 | 45,328 |
| 5 | 0,979 | 9,992 | 19,030 | 30,001 |
| 8 | 0,952 | 11,846 | 31,782 | 44,580 |
| 12 | 0,793 | 10,538 | 20,146 | 31,477 |

The results showed in Table 1 and FIG. 5 clearly demonstrate that the methods of the invention permit to concentrate an aqueous suspension of microalgae while maintaining the integrity of the cell structure.

What is claimed is:

1. A method of concentrating an aqueous suspension of microalgae, comprising the step of passing said suspension through a tangential filtering device to partially remove water from said suspension without rupturing said microalgae, thereby obtaining a concentrated suspension of microalgae and filtered water.

2. A method according to claim 1, wherein the suspension prior to being concentrated has a concentration ranging from 1 to 500×10⁶ cells/mL.

3. A method according to claim 2, wherein the concentration of said suspension ranges from 1×10⁶ to 50×10⁶ cells/mL.

4. A method according to claim 1, wherein the concentrated suspension obtained has a concentration ranging from 2 to 30×10¹⁰ cells/mL.

5. A method according to claim 4, wherein the concentration of said concentrated suspension ranges from 2×10⁶ to 10×10¹⁰ to cells/mL.

6. A method according to claim 1, wherein the concentrated suspension obtained is from 2 to 1000 times more concentrated than the suspension prior to concentration.

7. A method according to claim 6, wherein the concentrated suspension is from 100 to 800 times more concentrated than the suspension prior to concentration.

8. A method according to claim 1, wherein the microalgae in the concentrated suspension obtained are alive.

9. A method according to claim 1, wherein said tangential filtering device comprises a cartridge containing a plurality of spaced-apart parallel tubular members and wherein said tubular members have porous walls with pores of a predetermined molecular weight cut-off.

10. A method according to claim 9, wherein said tubular members are hollow fibers.

11. A method according to claim 9, wherein the molecular weight cut-off of said pores ranges from 1000 to 100000 Daltons.

12. A method according to claim 11, wherein the molecular weight cut-off of said pores ranges from 5000 to 20000 Daltons.

13. A method according to claim 1, wherein said tangential filtering device comprise a plurality of tangential filtration cartridges arranged in fluid flow communication with one another or in parallel relationship to one another.

14. A method according to claim 13, wherein said tangential filtration cartridges each contain a plurality of spaced-apart parallel tubular members and wherein said tubular members have porous walls with pores of a predetermined molecular weight cut-off.

15. A method according to claim 1, wherein the microalgae are selected from the group consisting of non-motile unicellular algae, flagellates, diatoms and blue-green algae.

16. A method according to claim 1, wherein the microalgae in the concentrated suspension obtained have a lipidic content which is stable for at least 12 days.

17. A method according to claim 1, wherein the microalgae in the concentrated suspension obtained have a phospholipid content which is stable for at least 12 days.

18. A method according to claim 1, wherein the microalgae in the concentrated suspension obtained have a cholesterol content which is stable for at least 12 days.

19. A method according to claim 1, wherein the microalgae in the concentrated suspension obtained have a reproductive potential which is maintained for a period of at least 25 days.

20. A method of producing a concentrated suspension of microalgae, comprising the steps of:
a) providing a reservoir containing an aqueous suspension of microalgae, and a tangential filtering device in fluid flow communication with said reservoir;
b) passing the suspension from said reservoir through said tangential filtering device to partially remove water from said suspension without rupturing said microalgae, thereby obtaining said concentrated suspension of microalgae and filtered water; and
c) recovering said concentrated suspension of microalgae.

21. A method according to claim 20, wherein said method further includes prior to step (c):
b′) recycling the concentrated suspension obtained in step (b) to said reservoir and then repeating step (b).

22. A method according to claim 21, wherein step (b′) is repeated until the suspension obtained reaches a desired concentration.

23. A method according to claim 22, wherein the desired concentration ranges from 1×10⁶ to 30×10¹⁰ cells/mL.

24. A method according to claim 22, wherein the desired concentration is from 4 to 1000 times higher than the concentration of the suspension used in step (a).

25. A method according to claim 24, wherein the desired concentration is from 100 to 800 times higher than the concentration of the suspension used in step (a).

26. A method according to claim 21, wherein a fresh suspension of microalgae is added into said reservoir during step (b) or (b′).

27. A method according to claim 22, wherein said method is a continuous method.

28. A method according to claim 20, wherein said tangential filtering device comprises a cartridge containing a plurality of spaced-apart parallel tubular members and wherein said tubular members have porous walls with pores of a predetermined molecular weight cut-off.

29. A method according to claim 28, wherein said tubular members are hollow fibers.

30. A method according to claim 20, wherein the microalgae in the concentrated suspension obtained are alive.

31. A method according to claim 1, used in a system for feeding marine organisms.

32. A method according to claim 1, used in a system for producing microalgae as food for marine organisms.

33. A method according claim 1, used in a system for producing microalgae as a health food.

34. A method according to claim 1, used in a system for producing microalgae as a biofuel.

35. A method according to claim 1, used in a system for producing microalgae for pharmaceutical use.

36. A method according to claim 1, used in a system for producing microalgae for extracting and/or isolating bioactive molecules.

37. An apparatus for concentrating an aqueous suspension of microalgae, comprising:
a reservoir dimensioned to contain the suspension of microalgae to be concentrated;
a tangential filtering device in fluid flow communication with said reservoir for partially removing water from said suspension without rupturing said microalgae; and
a pump for passing said suspension from said reservoir through said tangential filtering device, thereby obtaining a concentrated suspension of microalgae and filtered water.

38. An apparatus according to claim 37, wherein said tangential filtering device comprises a cartridge containing a plurality of spaced-apart parallel tubular members and wherein said tubular members have porous walls with pores of a predetermined molecular weight cut-off.

39. An apparatus according to claim 37, wherein said tangential filtering device comprise a plurality of tangential filtration cartridges arranged in fluid flow communication with one another or in parallel relationship to one another.

40. An apparatus according to claim 39, wherein said tangential filtration cartridges each contain a plurality of spaced-apart parallel tubular members and wherein said tubular members have porous walls with pores of a predetermined molecular weight cut-off.

41. An apparatus according to claim 38, wherein the molecular weight cut-off of said pores ranges from 1000 to 100000 Daltons.

42. An apparatus according to claim 41, wherein the molecular weight cut-off of said pores ranges from 5000 to 20000 Daltons.

43. An apparatus according to claim 38, wherein said tubular members are hollow fibers.

44. An apparatus according to claim 38, wherein said cartridge has a feed inlet for receiving the suspension of microalgae to be concentrated, a first outlet for discharging the filtered water and a second outlet for discharging the concentrated suspension of microalgae, and wherein said tubular members define therebetween a space in fluid flow communication with said first outlet, each said tubular member having an inlet in fluid flow communication with said feed inlet and an outlet in fluid flow communication with said second outlet.

45. An apparatus according to claim 44, wherein said second outlet is connected to said reservoir by a first conduit for recycling the concentrated suspension discharged from said cartridge.

46. An apparatus according to claim 44, wherein said feed inlet is connected to said reservoir by a second conduit.

47. An apparatus according to claim 44, wherein said first outlet is connected to a drain by another conduit.

48. An apparatus according to claim 46, wherein said second conduit is provided with a drain for emptying said reservoir.

49. An apparatus according to claim 46, wherein said second conduit is provided with a drain for emptying said cartridge.

50. An apparatus according to claim 45, wherein said first conduit is provided with a flow control device for controlling the flow rate of the concentrated suspension discharged from said cartridge.

51. An apparatus according to claim 46, wherein said second conduit is provided with a flow control device for controlling the flow rate of the suspension passing through said tangential filtering device.
